(19) United States
(12) Patent Application Publication
(10) Pub. No.: US 2021/0090715 A1
(43) Pub. Date: Mar. 25, 2021

(54) SYNTHETIC IMAGE GENERATION

(71) Applicant: INTERNATIONAL BUSINESS MACHINES CORPORATION, Armonk, NY (US)

(72) Inventor: Henry Feldman, Cambridge, MA (US)

(21) Appl. No.: 16/575,678

(22) Filed: Sep. 19, 2019

(51) Int. Cl. G16H 30/20; G10L 15/26; G10L 15/18; G06F 17/22

(52) U.S. Cl. CPC G16H 30/20; G10L 15/265; G06N 20/00; G06F 17/2235; G10L 15/18

(57) ABSTRACT

An embodiment of the invention may include a method, computer program product and computer system for the generation of synthetic images to enhance a diagnostic imaging order. The method, computer system, and computer program product may include receiving a verbal description of a clinical finding with respect to an anatomical part of a patient. The computing device may generate text from the received verbal description. The computing device may then identify a clinical finding in the text, the clinical finding describing a portion of an anatomical part of the patient at a particular location within the anatomical part. The computing device may also generate a synthetic image of the anatomical part including a mark indicating the portion at the particular location within the anatomical part of the clinical finding. Additionally, the computing device may be capable of generating a textual clinical order based on the received verbal description.

### BACKGROUND

The present invention relates generally to a method, system, and computer program for generating a synthetic image. More particularly, the present invention relates to a method, system, and computer program for generating a synthetic image of an anatomical part to enhance a diagnostic imaging order.

Medical imaging includes techniques and processes to create visual representations of the interior of a body for clinical analysis and medical intervention. Diagnostic imaging specialists rely on the information given to them from an ordering clinician to determine what portion of the patient's body needs to be imaged. Generally, an ordering clinician, e.g., a primary care physician, who physically examines the patient, completes a templated text order for a diagnostic imaging specialist to read prior to imaging. The order will give the imaging specialist enough information to image the general area of the patient being examined by the ordering clinician. The imaging specialist will send the image or images to be interpreted by a medical image interpreting specialist, e.g. a radiologist.

### BRIEF SUMMARY

An embodiment of the invention may include a method, computer program product and computer system for the generation of synthetic images to enhance a diagnostic imaging order. The method, computer system, and computer program product may include receiving a verbal description of a clinical finding with respect to an anatomical part of a patient. The computing device may generate text from the received verbal description. The computing device may then identify a clinical finding in the text, the clinical finding describing a portion of an anatomical part of the patient at a particular location within the anatomical part. The computing device may also generate a synthetic image of the anatomical part including a mark indicating the portion at the particular location within the anatomical part of the clinical finding. Additionally, the computing device may be capable of generating a textual clinical order based on the received verbal description. The computing device may generate a caption for the synthetic image of the anatomical part. The caption may be a textual description of the anatomical part and the clinical finding. The computing device may generate a link for the synthetic image of the anatomical part in the generated textual clinical order. The computing device may submit the generated textual clinical order along with the synthetic image of the anatomical part to a diagnostic image specialist using a computerized provider order entry in an electronic health record system.

### BRIEF DESCRIPTION OF THE DRAWINGS

FIG. 1a illustrates a system for synthetic image generation, in accordance to at least one embodiment.

FIG. 1b illustrates example operating modules of the synthetic image generation program of FIG. 1a.

FIG. 1c illustrates an example synthetic image created by the synthetic image generation program of FIG. 1a.

FIG. 2 is a flowchart illustrating an example method of generic anatomical image generation, in accordance with an embodiment of the invention.

FIG. 3 is a block diagram depicting the hardware components of the synthetic image generation system of FIG. 1, in accordance with an embodiment of the invention.

FIG. 4 illustrates a cloud computing environment, in accordance with an embodiment of the invention.

FIG. 5 illustrates a set of functional abstraction layers provided by the cloud computing environment of FIG. 4, in accordance with an embodiment of the invention.

### DETAILED DESCRIPTION

Embodiments of the present invention will now be described in detail with reference to the accompanying Figures.

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present invention provide a method, computer program, and computer system for generation of a synthetic image of an anatomical area from a verbal description of a physical finding. The verbal description of the physical finding may be from an ordering clinician. The synthetic anatomical image may be linked to a diagnostic order. Diagnostic imaging (screening) is mostly dependent on a computerized clinician order entry (i.e. CPOE) input to an electronic health record (i.e. EHR) by a clinician of the patient. These orders are often scantily filled out such that much of the ordering clinician's physical findings are not conveyed to the imaging and image interpreting specialists. The image interpreting specialist, therefore, may receive very limited information in text format as to the finding the ordering clinician wishes to have examined. In addition to limited textual information, the image interpreting specialist does not have physical access to the patient to perform a physical examination themselves. The lack of information received by the image interpreting specialist may result in significant delays in interpretation and may potentially result in missed diagnosis.

Embodiments of the present invention utilize methods of speech analysis to interpret verbal physical findings, e.g., of an ordering clinician, to generate a synthetic image of an anatomical area that may be linked or attached to a diagnostic study order to convey physical findings to an image interpreting clinician with high fidelity. The present invention allows the ordering clinician (e.g., examining clinician) to verbally describe their findings while simultaneously examining the patient, rather than have to type it in from recall as required by current systems. The textual description generated by the synthetic generation program may be linked to the diagnostic order. The present invention improves current technology by additionally linking a generated three-dimensional synthetic anatomical image to an image order. The generated synthetic image illustrates the anatomic location of the physical finding described by the ordering clinician along with any other details of the physical finding such as location and depth of the finding, size, and the nature of the finding, etc. This information provides the image interpreting specialist with a more informed starting point on which to concentrate, such as providing approximate dimensions of height, width, and depth relative to anatomical landmarks on the patient. This information frees the ordering clinician from having to know specific anatomical terms in the image interpreting specialists' domain as the image interpreting specialist will be able to rely on the generated synthetic image instead of solely on the ordering clinician's written order.

Embodiments of the present invention aim to improve the speed, efficiency, and accuracy of diagnostic orders by generating and linking synthetic anatomical images to the diagnostic orders. Additionally, embodiments of the present invention aim to reduce unnecessary phone calls or meetings between an ordering clinician and an image interpreting specialist by clearly indicating the anatomical position on the patient of the physical findings of interest to the ordering specialist.

Reference will now be made in detail to the embodiments of the present invention, examples of which are illustrated in the accompanying drawings, wherein like reference numerals refer to like elements throughout. Embodiments of the invention are generally directed to a system for generating a synthetic image of an anatomical area, based on a clinician's physical examination of a patient, to enhance a diagnostic imaging order. While embodiments are described with reference to an example of a breast area of human body, the principles of the invention may be used with any anatomical area of a patient, human or animal, requiring imaging, e.g., a foot, shoulder, or knee.

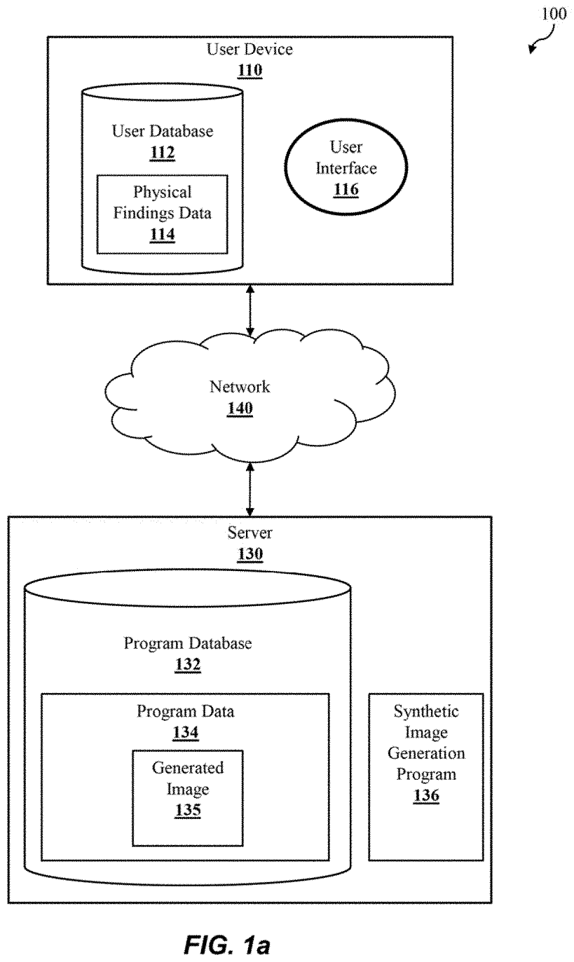

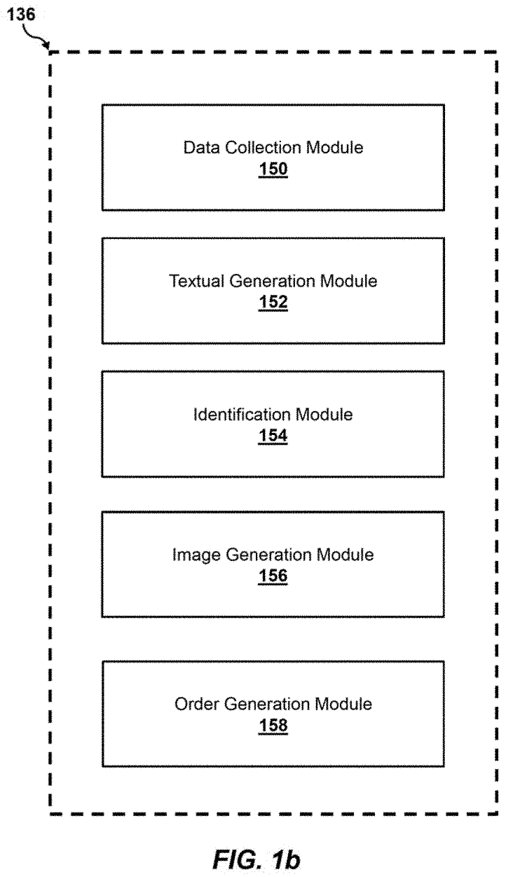

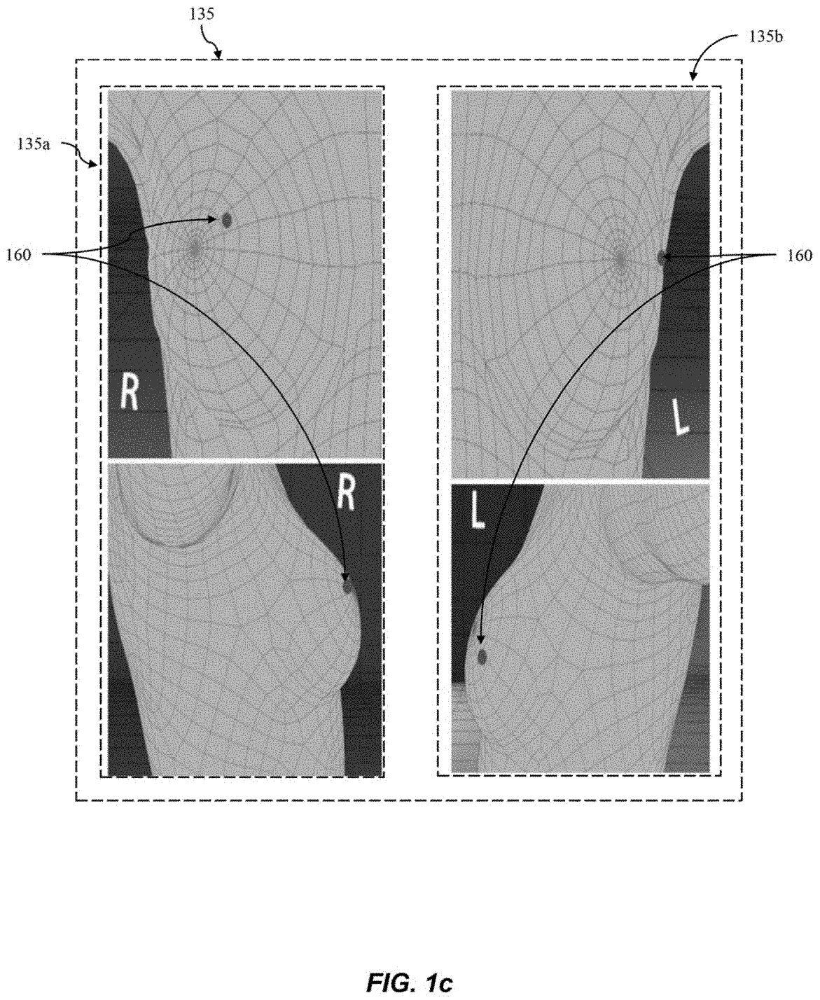

FIG. 1a illustrates a synthetic image generation system 100, in accordance with an embodiment of this invention. In an example embodiment, the synthetic image generation system 100 includes a user device 110 and a server 130, interconnected via a network 140.

In the example embodiment, the network 140 is the Internet, representing a worldwide collection of networks and gateways to support communications between devices connected to the Internet. The network 140 may include, for example, wired, wireless, or fiber optic connections. In other embodiments, the network 140 may be implemented as an intranet, a local area network (LAN), a wide area network (WAN), or a personal area network (PAN). In general, the network 140 can be any combination of connections and protocols that will support communications between the user device 110 and the server 130.

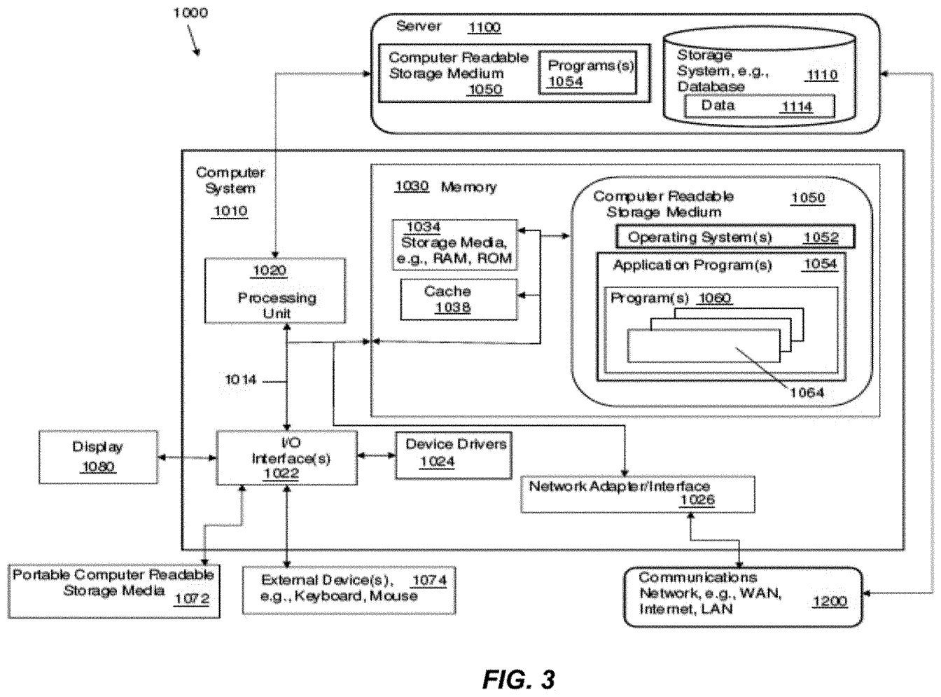

The user device 110 may include the user database 112 and the user interface 116. In the example embodiment, the user device 110 may be a cellphone, desktop computer, notebook, a laptop computer, a tablet computer, a thin client, a microphone, or any other electronic device or computing system capable of collecting, compiling, organizing, and storing audio, visual, or textual content and receiving and sending that content to and from other computing devices such as the server 130 via the network 140. The user device 110 may receive input, such as but not limited to, textual, visual, or audio input, from a physical input device, such as, but not limited to, camera, a microphone, a keyboard, etc. While only a single user device 110 is depicted, it can be appreciated that any number of user devices may be part of the generated synthetic image generation system 100. The user device 110 is described in more detail with reference to FIG. 3.

The user database 112 may store the physical findings data 114. The user database 112 may be any storage media capable of storing data, such as, but not limited to, storage media resident in the server 130 and/or removeable storage media. For example, the user database 112 may be, but is not limited to, a hard drive, a solid state drive, a USB drive, or a memory card. The user database 112 is described in more detail below and with reference to FIG. 3.

The physical findings data 114 may include textual, visual, and/or audio data. The physical findings data 114 may include, for example, data obtained from an ordering clinician examining a patient such as, but not limited to, a verbal description of one or more physical findings, or a hand-drawn diagram of the patient indicating one or more physical findings. Further, the physical findings data 114 may include data identifying the source of the physical findings data 114 such as, but not limited to patient data, including but not limited to: patient name, patient age, patient sex, height, weight, body type, patient medical history, prior medical images (x-rays, cat scans, MRIs, etc.) of the patient, anatomical area to be imaged, location of physical finding, date of imaging, etc. In embodiments, the physical findings data 114 may also include data obtained from a diagnostic imaging specialist interpreting a diagnostic order received from the ordering clinician. For example, the physical findings data 114 may include, but is not limited to, a diagnosis, an indication of no findings, or an indication of an anomaly found.

In the example embodiment, the user interface 116 includes components used to receive input from a user on the user device 110 and transmit the input to the synthetic image generation program 136 residing on the server 130, or conversely to receive information from the synthetic image generation program 136. In an example embodiment, the user interface 116 may receive input, such as but not limited to, textual, visual, or audio input, from a physical input device, such as, but not limited to, a keypad, a microphone, a camera, etc. For example, the user interface 116 may receive audio input such as, but not limited to, a clinician's verbal findings during a patient exam indicating one or more physical findings, e.g. one or more areas of concern for diagnostic imaging. Further, the user interface 116 may receive visual input such as, but not limited to, a clinician's hand-drawn diagram of a patient indicating one or more physical findings indicating an area of concern or location of a physical finding for diagnostic imaging.

The server 130 may include the program database 132 and the synthetic image generation program 136. In the example embodiment, the server 130 may be a desktop computer, a notebook, a laptop computer, a tablet computer, a thin client, or any other electronic device or computing system capable of storing, compiling, and organizing, audio, visual, or textual content and receiving and sending that content to and from other computing devices, such as the user device 110 via the network 140. In some embodiments, the server 130 includes a collection of devices, or data sources, in order to collect the program data 134. The server 130 is described in more detail with reference to FIG. 3.

The program database 132 may store the program data 134. The program database 132 may be any storage media capable of storing data, such as, but not limited to, storage media resident in the server 130 and/or removeable storage media. For example, the program database 132 may be, but is not limited to, a hard drive, a solid state drive, a USB drive, or a memory card, etc. The program database 132 is described in more detail below and with reference to FIG. 3.

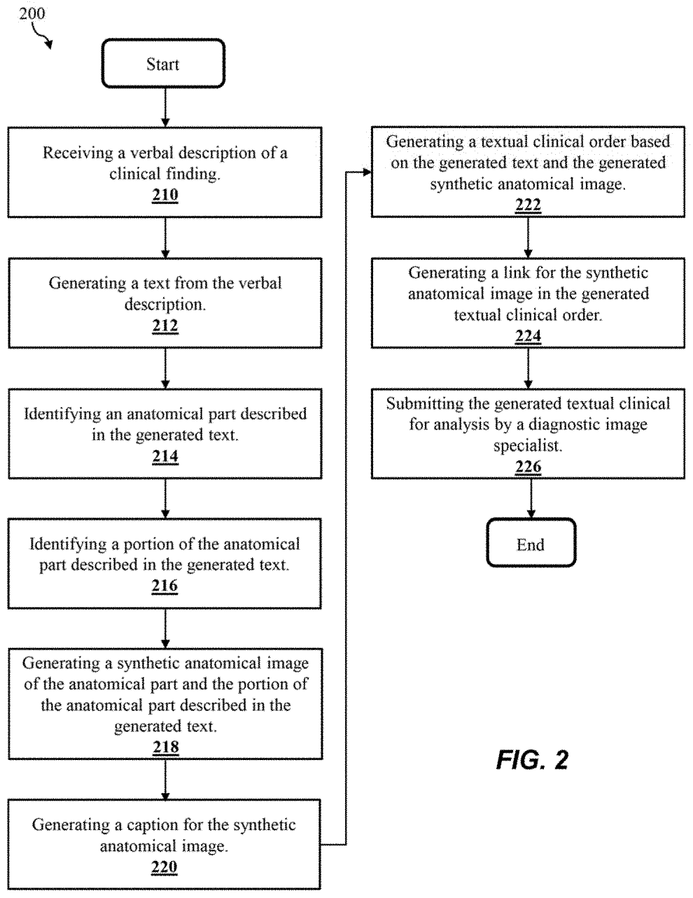

The program data 134 may be a collection of audiovisual content required by the synthetic image generation program 136 including, but not limited to, audio, visual, and textual content. The program data 134 may be, for example, but not limited to, the physical findings data 114 received and/or collected from the user device 110, and/or the generated image 135 generated by the synthetic image generation program 136. The generated image 135 is described in more detail below with reference to FIGS. 1b, 1c, and 2. Further, the program data 134 may include, but is not limited to, user data, patient data, imaging studies, and medical reports, etc. The program data 134 may include general anatomical data, such as anatomical landmarks of a human body, general descriptions and visual representations of anatomical areas and parts. The program data 134 is located on the server 130 and can be accessed via the network 140. In accordance with an embodiment of the invention, the program data 134 may be located on one or more a plurality of the server 130.

The synthetic image generation program 136 is a program capable of receiving the physical findings data 114 captured by the user device 110 and generating text from the received physical findings data 114 using machine learning techniques, which may include natural language processing. The synthetic image generation program 136 is then capable of identifying an anatomical part of a patient's body and a portion or location of the anatomical part of interest, e.g., having a possible abnormality, described within the generated text. Further, the synthetic image generation program 136 is capable of generating a generic image of the anatomical part including a representation of the anatomical portion of interest at the described location. A “generic image” of an anatomical part, as used herein, before the image is modified to include an anatomical portion of interest at the particular location is typically not be an actual image, or even one that approximates an actual image, of the anatomical portion referred to in the physical findings data. In various embodiments, the term “generic image” is used to refer to an image that may be a representative image of the anatomical part. In another embodiment of the invention, the generic image may be a prior medical image of the patient if such an image is located in the program data 134. Additionally, the synthetic image generation program 136 is capable of generating a textual clinical order based on the received physical findings data 114. The synthetic image generation program 136 is described in more detail below with reference to FIG. 1b.

FIG. 1b illustrates example modules of the synthetic image generation program 136. In an example embodiment, the synthetic image generation program 136 may include five modules: data collection module 150, textual generation module 152, identification module 154, image generation module 156, and order linkage module 158.

The data collection module 150 receives the physical findings data 114 captured from the user device 110. For example, but not limited to, the user device 110 may collect a verbal description of a physical finding from a microphone during an examination of a patient. The received verbal description of a physical finding may then be sent to the server 130 via the network 140 where it would be received by the data collection module 150 of the synthetic image generation program 136. In an embodiment of the invention, the physical findings data 114 may be stored in the program data 134 on the program database 132.

As an illustrative example, the user device 110 may capture speech of the following from an ordering clinician, Doctor A, “With reference to patient A, birthdate Jan. 1, 2011, it appears as though there is a hard mass on the inner quadrant of the right breast. It presents itself 2 o'clock at 3 cm from the nipple. Such hard mass may indicate breast cancer. I will order a full-field digital mammography.”

The textual generation module 152 generates text from the received physical findings data 114 of the user device 110. The textual generation module 152 may first utilize one or more machine learning techniques to analyze, interpret, and transcribe the physical findings data 114, which may be, audio, visual and/or textual data, into a textual description. Machine learning techniques may include, but are not limited to, deep artificial neural networks (i.e., DNN), recurrent neural networks (i.e., RNN), hidden Markov models (i.e., HMM), and Gaussian mixture models (i.e., GMM). As an example, the textual generation module 152 may use natural language processing to transcribe the received physical findings data 114 into a linguistically understood textual data form. The generated textual description may consist of the transcription of the interpreted physical findings data 114. In at least one embodiment, the textual description may include, but is not limited to, conversational terms (e.g., tumor), industry standard anatomical terms (e.g., neoplasm found within the Systemized Nomenclature of Medicine (i.e., SNOMED)), and an identifying code (e.g., C50.919). An identifying code, within the textual description, may be associated with data of a system used by physicians and other healthcare providers to classify and code all diagnoses, symptoms, and procedures recorded in conjunction with hospital care (e.g., International Statistic Classification of Diseases and Related Health Problems (i.e., ICD)). The plurality of identifying codes with the corresponding known diagnoses, symptoms, procedures, and hospital care may be found in the program data 134 of the server 130. In other embodiments, the plurality of identifying codes may be stored in the physical findings data 114.

In furthering the previous example, the textual generation module 152 analyzes the obtained verbal description of the Doctor A and generates the following textual description, “1 cm hard mass deep right inner quadrant at 2 o'clock position 3 cm from the areola of the right breast”.

The identification module 154 identifies the anatomical part discussed within the textual description. The identification module 154 may first obtain the generated textual description of the textual description module 154 from, as an example, the program data 134. Further, the identification module 154 is capable of analyzing and identifying the anatomical part described within the textual description. The anatomical parts identified within the textual description may be identified based on indexed or mapped anatomical terms, e.g. anatomical parts, to particular generic anatomical images stored in the program data 134. Identified anatomical parts may include terms associated with the location of the body that the order should focus on (e.g., the breast) in addition to more specific terms such as directional terms, terms associated with planes of the body or body cavities. Directional terms may include, but are not limited to, cranial, caudal, ventral, dorsal, medial, lateral, proximal, distal. Planes of the body may include, but are not limited to, the frontal plane, the lateral plane, the transverse plane, and the medial plane. The identification module 154 may extract the data associated with the anatomical part identified within the textual description utilizing one or more machine learning algorithms. Machine learning algorithms may include, but are not limited to, deep artificial neural networks (i.e., DNN), recurrent neural networks (i.e., RNN), hidden Markov models (i.e., HMM), and Gaussian mixture models (i.e., GMM). Subsets of such algorithms may include, natural language processing (i.e., NLP).

In at least one embodiment, wherein the textual description includes an identifying code, the identification module 154 may obtain the identifying code and parse through the plurality of identifying codes obtained from the program data 134 or the physical findings data 114 to obtain the anatomical part of the physical findings data 114 noted within the textual description. In such embodiments, the anatomical part of the physical finding may be found within the ICD database.

For example, the identification module 154 uses natural language processing of the textual description, to identify that the physical finding of Doctor A is on the right breast.

The identification module 154 identifies the portion of the anatomical part described in the text (i.e., the physical finding location, the finding location) discussed by the ordering clinician. The identification module 154 first obtains the generated textual description of the textual description module 154, from, as an example, the program data 134. Further, the identification module 154 is capable of analyzing and identifying the finding location described within the textual description. Identified finding locations may include terms associated with measurements or distances, and directions (e.g., 3 cm to the left, 5 cm to the right, 3 cm north). The identification module 154 may extract the finding location identified within the textual description utilizing one or more machine learning algorithms. Machine learning algorithms may include, but are not limited to, deep artificial neural networks (i.e., DNN), recurrent neural networks (i.e., RNN), hidden Markov models (i.e., HMM), and Gaussian mixture models (i.e., GMM). Subsets of such algorithms may include, natural language processing (i.e., NLP). The identification module 154 may also identify the clinical finding associated with the portion of the anatomical part described in the text utilizing similar machine learning techniques.

In at least one embodiment, wherein the textual description includes an identifying code, the identification module 154 may obtain the identifying code and parse through the plurality of identifying codes obtained from the program data 134 or the physical findings data 114 to determine the finding location of the physical findings data 114 noted within the textual description.

With regard to the previous example, the identification module 154 uses natural language processing of the textual description to identify that the physical finding described by Doctor A is located at 2 o' clock 3 cm from the nipple of the previously identified right breast.

The image generation module 156 generates a synthetic image of the identified anatomical part including the portion of the anatomical part. The image generation module 156 may first obtain a generic anatomical image of the identified anatomical part. A plurality of generic anatomical images may be stored in the program data 134. The generic images may be for example, but not limited to, three-dimensional or two-dimensional anatomical images. In other embodiments, the plurality of generic anatomical images may be stored in the physical findings data 114. Further, the generic images may contain a plurality of indexed or mapped anatomical parts associated with each generic image. For example, the generic image of a breast may have anatomical parts, such as, but not limited to, areola or nipple indexed or mapped to the generic image. The image generation module 156 may then render a marking of the identified clinical finding onto the identified portion of the generic anatomical part. Markings may include, but are not limited to, a basic shape covering the identified portion of the anatomic part (e.g., circle, square, triangle), a basic shape highlighting the identified portion of the anatomic part (e.g., a square around the identified portion of the anatomic part), a shape representing the shape of the clinical finding covering the identified portion of the anatomic part (e.g., a tumor shaped figure), a shape representing the shape of the identified clinical finding highlighting the portion of the anatomic part (e.g., a tumor shaped outline with an opaque fill), or an image of the physical findings data 114 (e.g., an image of a tumor). In embodiments, the image generation module 156 may interpret the portion of the anatomical part utilizing a machine learning algorithm, such as natural language processing, to allow the image generation module 156 to correctly place the representation on the anatomical structure on the generated image. An example marking is illustrated on the generated image 135 in FIG. 1c as markings 160. The plurality of generic anatomical images stored in the program data 134 may contain for example, but not limited to, a coordinate grid. Thus, the image generation module 156 may render a marking of the identified clinical finding onto the identified portion of the generic anatomical part based on the coordinate grid. For example, the identified clinical finding may be located “at the 2 o'clock position, 3 cm from the nipple,” and the image generation module 156 utilizing a machine learning algorithm may calculate the “o'clock” number as degrees, i.e. 20 degrees per “o'clock”, as 60 degrees from the nipple. Further, the image generation module 156 utilizing a machine learning algorithm may calculate the distance of “3 cm” using a scale such as, but not limited to 1 cm equals 15 pixels, etc.

In at least one embodiment, the image generation module 156 may generate a linked caption for the identified portion of the anatomic part of the generated image. In such embodiment, the linked caption may consist of a textual description of the anatomical part and the physical finding rendered onto the identified portion of the anatomical part.

In furthering the previous example, the image generation module 156 obtains a three-dimensional representation of the right breast. The image generation module 156 then places a circle with a 1 cm diameter onto the corresponding portion of the identified right breast of the three-dimensional representation, at the 2 o'clock position 3 cm from the nipple. In an alternative embodiment, the image generation module 156 may obtain a two-dimensional image.

The order generation module 158 generates a clinical imaging order. In at least one embodiment, the generated clinical order is based on the textually understood verbal description of the clinical findings. In at least one embodiment, the diagnostic order may include, but is not limited to, the name, age, date of birth, weight, and height of the patient, the symptoms/reason for the exam (e.g., hard mass), an identifying code (e.g.,C50.919), the name of the ordering physician (e.g., Doctor A), the ordered diagnostic imaging (e.g., FFDM), and the synthetic anatomical image generated by the image generation module 156. In embodiments, the order generation module 158 may compile the obtained data from the plurality of modules of the synthetic image generation program 136 to generate the diagnostic order. In other embodiments, the order generation module 158 may interpret the physical findings data 114 and extract the necessary information of the diagnostic order directly from the physical findings data 114 of the user database 112. In further embodiments, the order generation module 158 may receive input from the user interface 116 of the user device 110. In such embodiments, the ordering clinician may input information into the synthetic image generation program 136 via the communication network 140. In at least one embodiment, the order generation module 138 may generate the diagnostic order utilizing a combination of any of the aforementioned techniques. For example, the order generation module 158 may automatically interpret and transcribe the physical findings data 114 captured by the user device 110 into the necessary form of the generated diagnostic order, yet also prompt the ordering clinician to ensure all information is correct and add any missing information (e.g., the signature of the ordering clinician).

In at least one embodiment, the order generation module 158 may include the generated synthetic anatomical image in the generated diagnostic order of the ordering clinician. In other embodiments, the order linkage module 158 may store the generated synthetic anatomical image within the program data 134 of the server 130 or the physical findings data 114 of the user device 110. In such embodiments, the diagnostic order may include a link to the device in which the generated synthetic anatomical image is stored (e.g., a hypertext markup language link), wherein access to the link will allow the diagnostic imaging specialist to see the generated synthetic anatomical image. For example, the ordering clinician may utilize a picture archiving and communication system (i.e., PACS) to digitally store the image in addition to other study images and moreover, link the diagnostic order to the picture within the PACS. In further embodiments, the order linkage module 158 may utilize a blockchain structure to create an immutable record of generated synthetic anatomical image that may be managed by a cluster of computers (e.g., the computer of the ordering clinician, the computer of the diagnostic imaging specialist).

In furthering the previous example, the order generation module 158 may obtain the plurality of obtained and interpreted data to automatically generate a diagnostic order, as shown in Table 1, containing a HTML link to the generated synthetic anatomical image of the right breast. The generated synthetic anatomical image is represented with reference to 135a of FIG. 1c. Another example of a generated synthetic anatomical image is represented with reference to 135b of FIG. 1c.

TABLE 1
Diagnostic Order

| | | | | |
|---|---|---|---|---|
| Patient Name: Patient A | DOB: 1/1/11 | ICD Code: C50.919 | Ordering Clinician: Doctor A | Ordered Image: FFDM |

Anatomical Part: Right Breast
Portion of the Anatomical Part: 2 o'clock 3 cm from areola

Generated synthetic Anatomical Image: <a href = “. . . /html-link.htm”><img src =“patient.jpg” width = “82” height = “86”
title=“Patient A Generated synthetic Anatomical Image” alt=“PatientA”> </a>

In embodiments of the present invention, the synthetic image generation program 136 may store all obtained and generated data from the modules of the synthetic image generation program 136 to the electronic health record (i.e., EHR) of the patient.

In at least one embodiment, the synthetic image generation program 136 may include an opt-in/opt-out feature in which the user may select to opt-in to or opt-out of the collection of data associated with the specific user. The opt-in/opt-out feature may be changed at any time, and if the user opts in to the synthetic image generation program 136, then the user may be notified when the data collected is activated and the data collection has commenced. In some embodiments, the user may limit when data is collected, how the data is collected, what type of data is collected and the purposes for which the data collected is utilized. The user may also limit how and where the data is stored.

In at least one embodiment, the synthetic image generation program 136 may allow for the ordering clinician to review and make changes to the diagnostic order or the linked generated synthetic anatomical image before submission to the diagnostic imaging specialist. In such embodiments, the synthetic image generation program 136 may make use of the user interface 116 of the user device 110.

Referring to FIG. 2, a method 200 for generating, from a verbal description of a physical finding of an ordering clinician, a synthetic anatomical image linked to a diagnostic order, is depicted in accordance with an embodiment of the present invention.

Referring to block 210, the data collection module 150 receives a verbal description of a clinical finding, e.g., the physical findings data 114, captured from the user device 110. Physical findings data collection is described in more detail above with reference to the data collection module 150 of FIG. 1b.

Referring to block 212, the textual generation module 152 generates a textual interpretation of the received verbal description, e.g. the physical findings data 114. Textual generation is described in more detail above with reference to the textual generation module 152 of FIG. 1b.

Referring to block 214, the identification module 154 identifies an anatomical part described within the generated textual interpretation of the obtained physical findings data 114. Anatomical identification is described in more detail above with reference to the identification module 154 of FIG. 1b.

Referring to block 216, the identification module 154 identifies a portion of the identified anatomical part described within the generated textual interpretation of the obtained physical findings data 114. Anatomical identification is described in more detail above with reference to the identification module 154 of FIG. 1b.

Referring to block 218, the image generation module 156 generates synthetic anatomical image, representing the identified anatomical part and the portion of the anatomical part, the portion being designated with a marking. Anatomical image generation is described in more detail above with reference to the image generation module 156 of FIG. 1b.

Referring to block 220, the image generation module 156 generates a caption for the generated synthetic anatomical image. Caption generation is described in more detail above with reference to the image generation module 156 of FIG. 1b.

Referring to block 222, the order generation module 158 generates a textual clinical order. Clinical order generation is described in more detail above with reference to the order generation module 158 of FIG. 1b.

Referring to block 224, the order generation module 158 generates a link for the synthetic anatomical image in the generated textual clinical order. Link generation is described in more detail above with reference to the order generation module 158 of FIG. 1b.

Referring to block 226, the order generation module 158 submits the generated textual clinical order for analysis by a diagnostic image specialist. Order submission is described in more detail above with reference to the order generation module 158 of FIG. 1b.

Referring to FIG. 3, a system 1000 includes a computer system or computer 1010 shown in the form of a generic computing device. The method 200, for example, may be embodied in a program(s) 1060 (FIG. 3) embodied on a computer readable storage device, for example, generally referred to as memory 1030 and more specifically, computer readable storage medium 1050 as shown in FIG. 3. For example, memory 1030 can include storage media 1034 such as RAM (Random Access Memory) or ROM (Read Only Memory), and cache memory 1038. The program 1060 is executable by the processing unit or processor 1020 of the computer system 1010 (to execute program steps, code, or program code). Additional data storage may also be embodied as a database 1110 which can include data 1114. The computer system 1010 and the program 1060 shown in FIG. 3 are generic representations of a computer and program that may be local to a user, or provided as a remote service (for example, as a cloud based service), and may be provided in further examples, using a website accessible using the communications network 1200 (e.g., interacting with a network, the Internet, or cloud services). It is understood that the computer system 1010 also generically represents herein a computer device or a computer included in a device, such as a laptop or desktop computer, etc., or one or more servers, alone or as part of a datacenter. The computer system can include a network adapter/interface 1026, and an input/output (I/O) interface(s) 1022. The I/O interface 1022 allows for input and output of data with an external device 1074 that may be connected to the computer system. The network adapter/interface 1026 may provide communications between the computer system a network generically shown as the communications network 1200.

The computer 1010 may be described in the general context of computer system-executable instructions, such as program modules, being executed by a computer system. Generally, program modules may include routines, programs, objects, components, logic, data structures, and so on that perform particular tasks or implement particular abstract data types. The method steps and system components and techniques may be embodied in modules of the program 1060 for performing the tasks of each of the steps of the method and system. The modules are generically represented in FIG. 3 as program modules 1064. The program 1060 and program modules 1064 can execute specific steps, routines, sub-routines, instructions or code, of the program.

The methods of the present disclosure can be run locally on a device such as a mobile device, or can be run a service, for instance, on the server 1100 which may be remote and can be accessed using the communications network 1200. The program or executable instructions may also be offered as a service by a provider. The computer 1010 may be practiced in a distributed cloud computing environment where tasks are performed by remote processing devices that are linked through a communications network 1200. In a distributed cloud computing environment, program modules may be located in both local and remote computer system storage media including memory storage devices.

More specifically, as shown in FIG. 3, the system 1000 includes the computer system 1010 shown in the form of a general-purpose computing device with illustrative periphery devices. The components of the computer system 1010 may include, but are not limited to, one or more processors or processing units 1020, a system memory 1030, and a bus 1014 that couples various system components including system memory 1030 to processor 1020.

The bus 1014 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

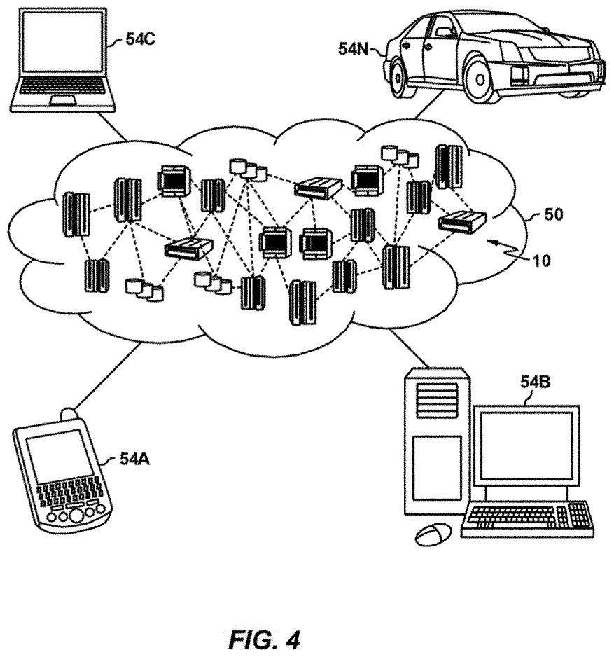

The computer 1010 can include a variety of computer readable media. Such media may be any available media that is accessible by the computer 1010 (e.g., computer system, or server), and can include both volatile and non-volatile media, as well as, removable and non-removable media. Computer memory 1030 can include additional computer readable media 1034 in the form of volatile memory, such as random access memory (RAM), and/or cache memory 1038. The computer 1010 may further include other removable/non-removable, volatile/non-volatile computer storage media, in one example, portable computer readable storage media 1072. In one embodiment, the computer readable storage medium 1050 can be provided for reading from and writing to a non-removable, non-volatile magnetic media. The computer readable storage medium 1050 can be embodied, for example, as a hard drive. Additional memory and data storage can be provided, for example, as the storage system 1110 (e.g., a database) for storing data 1114 and communicating with the processing unit 1020. The database can be stored on or be part of a server 1100. Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1014 by one or more data media interfaces. As will be further depicted and described below, memory 1030 may include at least one program product which can include one or more program modules that are configured to carry out the functions of embodiments of the present invention. As such, the computing device in FIG. 4 becomes specifically configured to implement mechanisms of the illustrative embodiments and specifically configured to perform the operations and generated the outputs of described herein for determining a route based on a user's preferred environmental experiences.

The method 200 (FIG. 2), for example, may be embodied in one or more computer programs, generically referred to as a program(s) 1060 and can be stored in memory 1030 in the computer readable storage medium 1050. The program 1060 can include program modules 1064. The program modules 1064 can generally carry out functions and/or methodologies of embodiments of the invention as described herein. For example, the program modules 1064 can include the modules 150-158 described above with reference to FIG. 1b. The one or more programs 1060 are stored in memory 1030 and are executable by the processing unit 1020. By way of example, the memory 1030 may store an operating system 1052, one or more application programs 1054, other program modules, and program data on the computer readable storage medium 1050. It is understood that the program 1060, and the operating system 1052 and the application program(s) 1054 stored on the computer readable storage medium 1050 are similarly executable by the processing unit 1020.

The computer 1010 may also communicate with one or more external devices 1074 such as a keyboard, a pointing device, a display 1080, etc.; one or more devices that enable a user to interact with the computer 1010; and/or any devices (e.g., network card, modem, etc.) that enables the computer 1010 to communicate with one or more other computing devices. Such communication can occur via the Input/Output (I/O) interfaces 1022. Still yet, the computer 1010 can communicate with one or more networks 1200 such as a local area network (LAN), a general wide area network (WAN), and/or a public network (e.g., the Internet) via network adapter/interface 1026. As depicted, network adapter 1026 communicates with the other components of the computer 1010 via bus 1014. It should be understood that although not shown, other hardware and/or software components could be used in conjunction with the computer 1010. Examples, include, but are not limited to: microcode, device drivers 1024, redundant processing units, external disk drive arrays, RAID systems, tape drives, and data archival storage systems, etc.

It is understood that a computer or a program running on the computer 1010 may communicate with a server, embodied as the server 1100, via one or more communications networks, embodied as the communications network 1200. The communications network 1200 may include transmission media and network links which include, for example, wireless, wired, or optical fiber, and routers, firewalls, switches, and gateway computers. The communications network may include connections, such as wire, wireless communication links, or fiber optic cables. A communications network may represent a worldwide collection of networks and gateways, such as the Internet, that use various protocols to communicate with one another, such as Lightweight Directory Access Protocol (LDAP), Transport Control Protocol/Internet Protocol (TCP/IP), Hypertext Transport Protocol (HTTP), Wireless Application Protocol (WAP), etc. A network may also include a number of different types of networks, such as, for example, an intranet, a local area network (LAN), or a wide area network (WAN).

In one example, a computer can use a network which may access a website on the Web (World Wide Web) using the Internet. In one embodiment, a computer 1010, including a mobile device, can use a communications system or network 1200 which can include the Internet, or a public switched telephone network (PSTN) for example, a cellular network. The PSTN may include telephone lines, fiber optic cables, microwave transmission links, cellular networks, and communications satellites. The Internet may facilitate numerous searching and texting techniques, for example, using a cell phone or laptop computer to send queries to search engines via text messages (SMS), Multimedia Messaging Service (MMS) (related to SMS), email, or a web browser. The search engine can retrieve search results, that is, links to websites, documents, or other downloadable data that correspond to the query, and similarly, provide the search results to the user via the device as, for example, a web page of search results.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

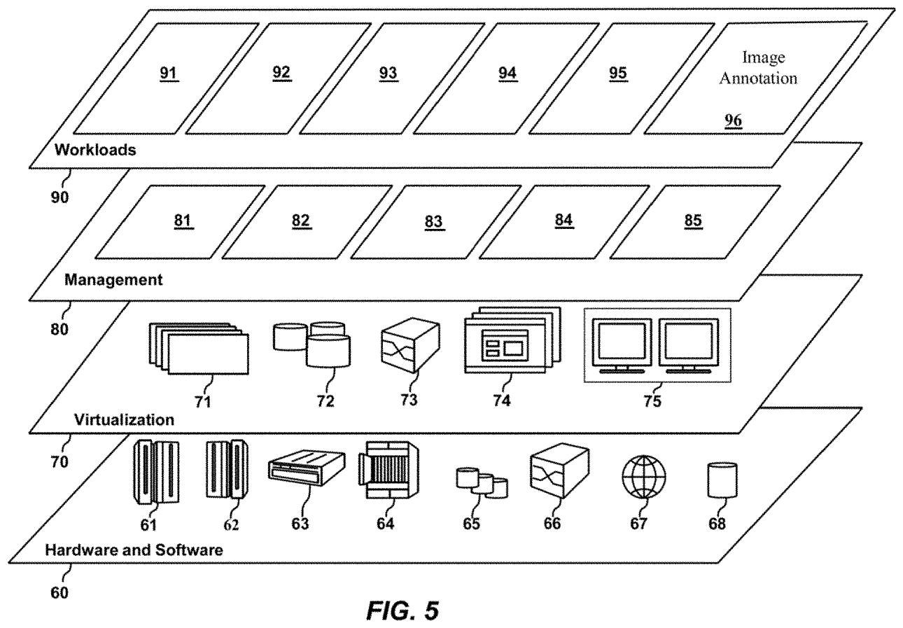

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and image generation 96.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While steps of the disclosed method and components of the disclosed systems and environments have been sequentially or serially identified using numbers and letters, such numbering or lettering is not an indication that such steps must be performed in the order recited, and is merely provided to facilitate clear referencing of the method's steps. Furthermore, steps of the method may be performed in parallel to perform their described functionality.

1. A method for the generation of synthetic images to enhance a diagnostic imaging order, the method comprising:
receiving, by a computer, a verbal description of a clinical finding with respect to an anatomical part of a patient;
generating, by the computer, text from the verbal description using natural language processing;
identifying, by the computer, a clinical finding in the text, the clinical finding describing a portion of an anatomical part of the patient at a particular location within the anatomical part, and the particular location interpreted using machine learning;
generating, by the computer, a synthetic image of the anatomical part, the synthetic image including a mark indicating the portion at the particular location within the anatomical part of the clinical finding; and
generating, by the computer, a textual clinical order based on verbal description of a clinical finding.

2. A method as in claim 1, further comprising:
generating, by the computer, a caption for the synthetic image of the anatomical part, the caption comprising a textual description of the anatomical part and the clinical finding.

3. The method as in claim 1, further comprising:
generating, by the computer, a link for the synthetic image of the anatomical part in the generated textual clinical order.

4. The method as in claim 1, further comprising:
submitting, by the computer, the generated textual clinical order along with the synthetic image of the anatomical part to a diagnostic image specialist using a computerized provider order entry in an electronic health record system.

5. The method of claim 1, wherein the synthetic image of the anatomical part is a 3-dimensional image of the identified anatomical part, the identified portion of the anatomical part, and the identified clinical finding in the identified portion of the anatomical part.

6. The method of claim 1, wherein identifying, by the computer, the clinical finding in the identified portion of the anatomical part, further comprises:
receiving the verbal description from an examiner during an examination of the patient, the verbal description of the clinical finding describing a portion of the anatomical part in which the clinical finding is located; and
utilizing natural language processing, identifying the clinical finding within the received verbal description from the examiner

7. A computer system for the generation of synthetic images to enhance a diagnostic imaging order comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
receiving, by a computer, a verbal description of a clinical finding with respect to an anatomical part of a patient;
generating, by the computer, text from the verbal description using natural language processing;
identifying, by the computer, a clinical finding in the text, the clinical finding describing a portion of an anatomical part of the patient at a particular location within the anatomical part, and the particular location interpreted using machine learning;
generating, by the computer, a synthetic image of the anatomical part, the synthetic image including a mark indicating the portion at the particular location within the anatomical part of the clinical finding; and
generating, by the computer, a textual clinical order based on verbal description of a clinical finding.

8. The computer system as in claim 7, further comprising:
generating, by the computer, a caption for the synthetic image of the anatomical part, the caption comprising a textual description of the anatomical part and the clinical finding.

9. The computer system as in claim 7, further comprising:
generating, by the computer, a link for the synthetic image of the anatomical part in the generated textual clinical order.

10. The computer system as in claim 7, further comprising:
submitting, by the computer, the generated textual clinical order along with the synthetic image of the anatomical part to a diagnostic image specialist using a computerized provider order entry in an electronic health record system.

11. The computer system as in claim 7, wherein the synthetic image of the anatomical part is a 3-dimensional image of the identified anatomical part, the identified portion of the anatomical part, and the identified clinical finding in the identified portion of the anatomical part.

12. The computer system as in claim 7, wherein identifying, by the computer, the clinical finding in the identified portion of the anatomical part, further comprises:
receiving the verbal description from an examiner during an examination of the patient, the verbal description of the clinical finding describing a portion of the anatomical part in which the clinical finding is located; and
utilizing natural language processing, identifying the clinical finding within the received verbal description from the examiner

13. A computer program product for the generation of synthetic images to a diagnostic imaging order comprising:
one or more computer-readable storage media and program instructions stored on at least one of the one or more tangible storage media, the program instructions executable by a processor to cause the processor to perform a method comprising:
receiving, by a computer, a verbal description of a clinical finding with respect to an anatomical part of a patient;
generating, by the computer, text from the verbal description using natural language processing;
identifying, by the computer, a clinical finding in the text, the clinical finding describing a portion of an anatomical part of the patient at a particular location within the anatomical part, and the particular location interpreted using machine learning;
generating, by the computer, a synthetic image of the anatomical part, the synthetic image including a mark indicating the portion at the particular location within the anatomical part of the clinical finding; and
generating, by the computer, a textual clinical order based on verbal description of a clinical finding.

14. The computer program product as in claim 13, further comprising:
generating, by the computer, a caption for the synthetic image of the anatomical part, the caption comprising a textual description of the anatomical part and the clinical finding.

15. The computer program product as in claim 13, further comprising:
generating, by the computer, a link for the synthetic image of the anatomical part in the generated textual clinical order.

16. The computer program product as in claim 13, further comprising:
submitting, by the computer, the generated textual clinical order along with the synthetic image of the anatomical part to a diagnostic image specialist using a computerized provider order entry in an electronic health record system.

17. The computer program product as in claim 13, wherein the synthetic image of the anatomical part is a 3-dimensional image of the identified anatomical part, the identified portion of the anatomical part, and the identified clinical finding in the identified portion of the anatomical part.

18. The computer program product as in claim 13, wherein identifying, by the computer, the clinical finding in the identified portion of the anatomical part, further comprises:
receiving the verbal description from an examiner during an examination of the patient, the verbal description of the clinical finding describing a portion of the anatomical part in which the clinical finding is located; and
utilizing natural language processing, identifying the clinical finding within the received verbal description from the examiner.
